(19) United States
(12) Patent Application Publication
(10) Pub. No.: US 2008/0208112 A1
(43) Pub. Date: Aug. 28, 2008

(54) Nasal Secretion Aspiration Device

(75) Inventor: Jóse Bensoussan, Beverly Hills, CA (US)

Correspondence Address: CANTOR COLBURN, LLP, 20 Church Street, 22nd Floor, Hartford, CT, 06103, US

(73) Assignee: UBIMED L.L.C., MIAMI, FL (US)

(21) Appl. No.: 11/718,926

(22) PCT Filed: Nov. 7, 2005
(86) PCT No.: PCT/FR05/02770

§ 371 Date: Sep. 19, 2007

(30) Foreign Application Priority Data
Nov. 9, 2004 (FR) 0411917

(51) Int. Cl. A61M 1/00

(52) U.S. Cl. 604/35

(57) ABSTRACT

The invention relates to a nasal secretion aspiration device. The inventive device consists of: a hollow body (3) which defines a vacuum chamber (9) that can be connected to a vacuum source (10), said chamber (9) comprising an opening (13); and a nasal end piece (2) comprising a first open end (5) which is designed to be inserted into a person's nose and a second open end (7) which is equipped with means for fixing same in a leak-tight, removable manner in the opening (13) of the hollow body (3). According to the invention, the second end (7) is sealed by a non-porous deformable element (8) under a vacuum created in the vacuum chamber (9) by the vacuum source (10).

The present invention relates to a nasal secretion aspiration device.

This device may be used for any person having difficulties with blowing their nose.

However, it is more particularly intended for blowing the noses of young children or infants who do not know how to blow their noses on their own, apart from sneezing or swallowing their nasal secretions through rear passages.

### BACKGROUND OF THE INVENTION

In the case of excessive production, the nasal secretions block up the nasal passages, causing great respiratory difficulties for young children and infants, given that said young children and infants breathe spontaneously through the nose.

Moreover, the Eustachian tube which isolates the cavity of the middle ear is very short in infants, a nasal blockage therefore promoting the multiplication of acute middle ear infections which are able to turn into chronic middle ear infections with effusion in the case of a chronic blockage of the nasal passages, with secondary hypertrophy of the adenoidal tissues.

Moreover, rhinopharyngeal infections are passed on to the tracheo-bronchial tree by inhalation.

The decongestion of the nasal passages of infants and young children is, therefore, essential from the hygienic and medical point of view, as an indispensable complement to washing the nasal passages with saline.

The use of antibiotic nasal solutions is strongly discouraged for infants and young children to avoid the increase in bacterial resistance.

It is therefore known to use a nasal secretion aspiration device for young children and infants.

### DESCRIPTION OF THE PRIOR ART

A plurality of types of device are known and they generally comprise a nasal end piece connected to a vacuum source.

A first type of known device consists of a rubber syringe connected to a nasal end piece.

This device consists in pressing on the rubber syringe in order to empty it of its air, then placing the nasal end piece in the nasal cavity of an individual. Then, by releasing the pressure on the syringe, said syringe readopts its original shape and generates a vacuum which allows the nasal secretions to be aspirated.

This system generates a violent, very brief vacuum which may be unpleasant for an infant or a young child.

For reasons of hygiene, it is necessary to clean and sterilize the syringe and the nasal end piece after each use of the device, the device not being for single use.

More specifically, the shape of the syringe makes complete cleaning and sterilization of the device difficult.

A second type of known device consists of a mouth end piece connected by a flexible tube to a reservoir connected to a nasal end piece.

This device consists in placing the nasal end piece in the nasal cavity of an individual and then aspirating the nasal secretions through the mouth end piece.

This type of device is difficult to use as it is slow to implement on an infant or a young child who has a tendency to struggle.

Moreover, the nasal secretions frequently rise up in the mouth end piece and are able to contaminate the person who is aspirating.

As before, the device has to be cleaned and sterilized after each use.

A third type of known device consists of a nasal end piece with a reservoir connected to an electric pump.

This device consists in placing the nasal end piece in the nasal cavity of an individual and generating a vacuum in the reservoir by means of the electric pump, resulting in the aspiration of the nasal secretions.

As before, the device has to be cleaned and sterilized after each use, said device not being for single use.

Moreover, in this type of device, the reservoir is not isolated from the electric pump, as a result of which the nasal secretions frequently rise up in the electric pump which is then contaminated and may jam.

The present invention, therefore, aims to remedy these drawbacks.

### SUMMARY OF THE INVENTION

The technical problem underlying the invention is to produce a nasal secretion aspiration device which does not require cleaning and sterilization after each use.

To this end, the invention relates to a nasal secretion aspiration device, characterized in that it comprises:
a hollow body defining a vacuum chamber able to be connected to a vacuum source, the vacuum chamber comprising an opening,
and a nasal end piece comprising a first open end adapted to the nostril, intended to be introduced into the nose of an individual and a second open end equipped with means for fixing in a leak-tight, removable manner in the opening of the hollow body, this second end being sealed by a non-porous element which may be deformed or displaced under the action of a vacuum created in the vacuum chamber by the vacuum source.

Thus, under the action of a vacuum created in the vacuum chamber, a vacuum is created in the nasal end piece by deformation or displacement of the non-porous deformable or displaceable element. This vacuum in the nasal end piece ensures the aspiration of the secretions.

As a result, during use of the device, the secretions are maintained in the nasal end piece forming the reservoir which is, for reasons of hygiene, removed and discarded after each use.

Thus, the hollow body and the vacuum source are not contaminated by the nasal secretions and do not have to be cleaned and sterilized after each use of the device.

In one embodiment of the invention, the element sealing the second end of the nasal end piece consists of an elastically deformable impermeable membrane, such as latex or polyvinyl chloride.

Advantageously, the element sealing the second end of the nasal end piece consists of impermeable material, such as coated paper folded in the manner of an accordion.

In one embodiment of the invention, the second end of the nasal end piece is equipped with a cylindrical and axial tubular extension, inside of which is housed a piston which is displaceable under the action of a vacuum between a position located in the region of the second end and a position remote therefrom.

Advantageously, the nasal end piece comprises two parts intended to be fixed to one another by nesting and in that the impermeable membrane is fixed to the nasal end piece by clamping between the two parts fixed by nesting.

In one embodiment of the invention, the impermeable membrane comprises a peripheral flange intended to be retained in a housing formed between the two parts fixed by nesting.

Advantageously, the peripheral flange is intended to cooperate with a retaining element formed on the external wall of one of the two parts forming the nasal end piece (2).

In one embodiment of the invention, the two parts forming the nasal element (2) comprise nesting means comprising a catch and a housing of corresponding shape in order to form a connection which may not be dismantled.

Advantageously, the nasal end piece comprises at its first end a non-return valve avoiding the emptying of the nasal end piece when a vacuum ceases to be exerted on the deformable or displaceable element sealing the second end.

In one embodiment of the invention, the non-return valve consists of a ball maintained in abutment under the action of a spring against the orifice of the first end, when no vacuum is created in the vacuum chamber.

Advantageously, the means for fixing the nasal end piece to the hollow body are implemented by screwing.

In one embodiment of the invention, the means for fixing the nasal end piece to the hollow body are catch, nesting or bayonet type means.

Advantageously, the hollow body contains an electric pump, driven by an electric motor, intended to create a vacuum in the vacuum chamber.

In one embodiment of the invention, the electric motor is supplied by batteries or an accumulator battery housed in the hollow body.

Advantageously, the vacuum chamber is equipped with an aspiration end piece for mounting an aspiration tube, for example an oral aspiration tube.

In one embodiment of the invention, the vacuum chamber is defined by a wall capable of limiting the deformation of the deformable element sealing the second end of the nasal end piece.

### BRIEF DESCRIPTION OF THE DRAWINGS

In any case, the invention will be easily understood by means of the following description, with reference to the indexed schematic drawing which shows, by way of non-limiting examples, a plurality of embodiments of said nasal secretion aspiration device.

FIG. 1 is a view in longitudinal section of a first embodiment of a nasal secretion aspiration device according to the invention, before assembly.

FIG. 2 is a view in cross section of a device according to the line II-II of FIG. 1.

FIG. 3 is a view in longitudinal section of a device according to FIG. 1 during operation.

FIG. 4 is a partial view in longitudinal section of a second embodiment of the invention.

FIG. 5 is a partial view in longitudinal section of a third embodiment of the invention.

FIG. 6 is a partial view in longitudinal section of a fourth embodiment of the invention.

FIG. 7 is a partial view in longitudinal section of a fifth embodiment of the invention.

### DESCRIPTION OF THE PREFERRED EMBODIMENTS

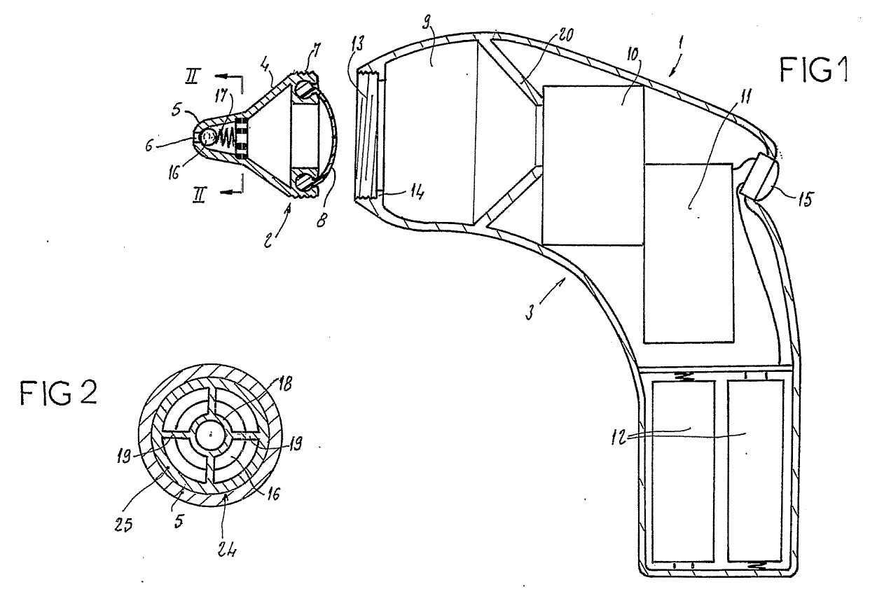

FIG. 1 shows a first embodiment of a nasal secretion aspiration device 1 according to the invention.

The nasal secretion aspiration device 1 comprises a nasal end piece 2 and a hollow body 3.

The nasal end piece 2 comprises a hollow central part 4 of truncated shape comprising, at its end of smaller section, a second hollow truncated part 5 of smaller diameter. The truncated part 5 comprises, at its end of smaller section, an orifice 6 and is suitable for being introduced into the nose of an individual.

The central truncated part 4 comprises, at its end of greater section, a collar 7 of which the external surface is defined by a portion with a cylindrical surface, the external surface of the collar being threaded.

The collar 7 is sealingly sealed by an elastically deformable impermeable membrane 8, preferably made of latex or polyvinyl chloride.

The fixing of the impermeable membrane 8 to the nasal end piece 2 is carried out by complementarity of shape.

According to further embodiments of the invention, the fixing of the impermeable membrane 8 to the nasal end piece 2 may advantageously be carried out by bonding or by welding or the two parts may even be molded or injection-molded together.

The hollow body 3 defines a vacuum chamber 9 connected to an electric pump 10 intended to create a vacuum in the vacuum chamber 9.

The electric pump 10 is driven by an electric motor 11 supplied by batteries 12 housed in the hollow body 3.

The electric motor 11 is associated with control means ensuring operation of the motor 11 with a time delay, thus the motor 11 is only able to operate continuously for a specific duration and this is in order to limit the vacuum produced and thus to avoid damage to the motor 11.

Beyond this duration, the motor 11 is stopped by the control means, and may only be restarted after a second specific duration.

The vacuum chamber 4 internally comprises a tapped opening 13 of complementary shape to that of the collar 7 of the nasal end piece 2, intended for screwing-on the nasal end piece 2.

The opening 13 of the hollow body 3 is defined, in the extension of its tapped portion, by a peripheral stop 14 intended to lock the nasal end piece 2 in position when screwed into the opening 13.

The nasal end piece 2 is consequently mounted in a removable manner on the hollow body 3.

A push button 15 is provided on the hollow body 3 in order to allow the supply of the electric motor 11 by the batteries 12 in its pushed-in position.

Thus, when pressing the push button 15, the electric pump 10 creates a vacuum in the vacuum chamber 9 which causes the deformation of the impermeable membrane 8.

The deformation of the impermeable membrane 8 transfers the vacuum into the nasal end piece 2, resulting in an aspiration of the nasal secretions.

In the truncated part 5, the nasal end piece 2 comprises a non-return valve consisting of a ball 16 maintained in abutment under the action of a spring 17 against the orifice 6 of the truncated part 5 when no vacuum is created in the vacuum chamber 9.

As shown in FIG. 2, the fixing zone of the spring 17 is formed by a separate part 24, forcibly mounted in the truncated part 5 of the nasal end piece 2. The separate part 24 is formed by a cylindrical and axial part 18 connected by four integral lugs 19 of the same length to a cylindrical part 25 of greater diameter.

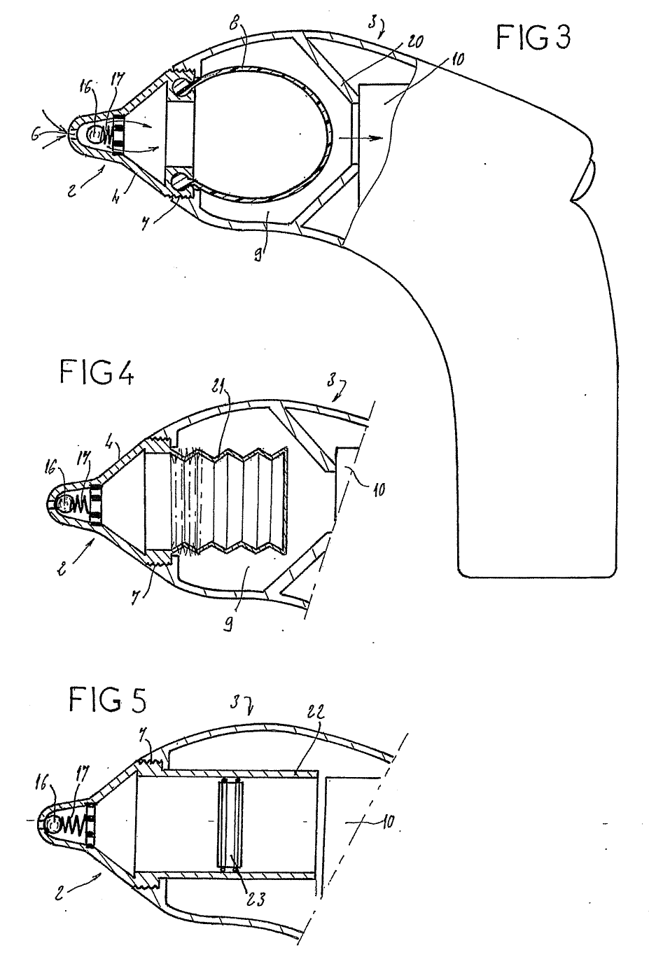

FIG. 3 shows the device according to FIG. 1 during operation.

When a vacuum is created in the vacuum chamber 9, the impermeable membrane 8 is deformed and transfers the vacuum to the nasal end piece 2.

A compression of the spring 17 results and, consequently, the ball 16 no longer seals the orifice 6 of the truncated part 5 of the nasal end piece 2, allowing the aspiration of the nasal secretions.

When the vacuum ceases to be exerted on the impermeable membrane 8, the ball 16, under the action of the spring 17, seals the orifice 6 of the truncated part 5 again, thus avoiding the emptying of the nasal end piece 2.

For reasons of hygiene, the nasal end piece 2 is preferably for single use.

After use of the nasal secretion aspiration device 1, it is therefore possible to unscrew the used nasal end piece 2 from the hollow body 3, and then to replace it with a new nasal end piece 2.

Thus the hollow body and the vacuum source are not contaminated by the nasal secretions and do not need to be cleaned and sterilized after each use of the device.

The vacuum chamber 9 is defined by a wall 20, allowing the connection between the vacuum chamber 9 and the electric pump 10, capable of limiting the deformation of the impermeable membrane 8.

FIG. 4 shows a second embodiment of the element sealing the collar 7 of the nasal end piece 2.

According to this embodiment of the invention, the collar 7 is sealingly sealed by a deformable element 21 consisting of impermeable material, such as coated paper, folded in the manner of an accordion.

The fixing of the deformable element 21 to the nasal end piece 2 is carried out by bonding.

FIG. 5 shows a third embodiment of the element sealing the collar 7 of the nasal end piece 2.

According to this embodiment of the invention, the collar 7 is equipped with a cylindrical and axial tubular extension 22, inside of which is housed a piston 23, which is displaceable under the action of a vacuum between a position located in the region of the collar 7 and a position remote therefrom.

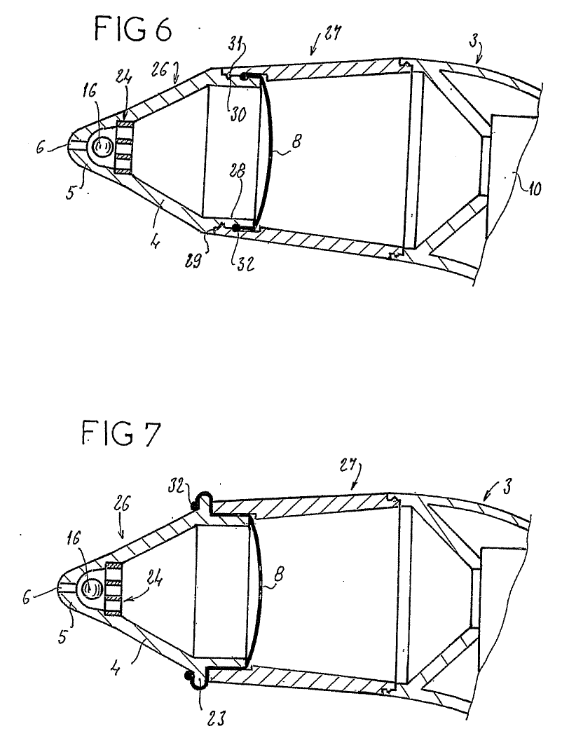

FIG. 6 shows a fourth embodiment of the nasal secretion aspiration device 1.

In this embodiment, the same elements bear the same reference numerals.

In contrast to the first embodiment, the nasal end piece 2 comprises two parts 26, 27 instead of one.

The first part 26 comprises, in a similar manner to the first embodiment, a central hollow part 4 of truncated shape, a second hollow truncated part 5, an orifice 6, a ball 16 and a part 24 forcibly mounted in the truncated part 5.

In this embodiment, in particular, the central hollow part 4 of truncated shape comprises at its end of greater section, a cylindrical and axial tubular extension 28 provided on its external surface with means for fixing to the second part 27.

The external diameter of the tubular extension 28 is less than the external diameter of the central part 4 at its end of greater section. As a result, a peripheral edge 29 is formed, acting as a stop when the first part 26 is nested in the second part 27.

The second part 27 is hollow and of substantially truncated shape. The second part 27 comprises a first open end equipped with means for fixing in a leak-tight, removable manner in the opening 13 of the hollow body 3 and a second end of complementary shape to that of the tubular extension 28, intended for the nesting of the first part 26.

A housing 30 is formed on the external surface of the tubular extension 28 and a catch 31 with a shape corresponding to that of the housing 30 is formed on the internal surface of the part 27, the catch 31 being intended to nest in the housing 30, these two elements forming nesting means in order to create a connection between the parts 26 and 27 which may not be dismantled.

The impermeable membrane 8 comprises a peripheral flange 32 intended to be retained in a housing formed between the two parts 26, 27 fixed by nesting in the region of the tubular extension 28.

The impermeable membrane 8 is thus fixed to the nasal end piece 2 by clamping between the two parts 26, 27 and ensures the seal between the two parts 26, 27.

According to this embodiment of the invention, in the truncated part 5, the nasal end piece 2 comprises a non-return valve solely consisting of the ball 16.

When a vacuum ceases to be exerted on the impermeable membrane 8, the ball 16 seals the orifice 6 of the truncated part 5, under the action of the pressure exerted by the impermeable membrane 8 which has the tendency to readopt its initial shape, thus avoiding the emptying of the nasal end piece 2.

After use of the nasal secretion aspiration device 1, it is thus possible to remove the used nasal end piece 2 from the hollow body 3, and then to replace it with a new nasal end piece 2.

According to this embodiment of the invention, during use of the nasal secretion aspiration device, the membrane 8 is deformed and flattened against the internal surface of the second part 27. As a result, during the dismantling of the nasal end piece 2, the membrane 8 is not compressed and the nasal secretions are not able to leave the nasal end piece 2, thus avoiding partial emptying of the nasal secretions.

FIG. 7 shows a fifth embodiment of the nasal secretion aspiration device 1.

According to this embodiment of the invention, the peripheral flange 32 of the impermeable membrane 8 is intended to cooperate with a retaining element 33 formed on the external wall of the first part 26, in the region of the end of greater section of the central part 4.

The two parts 26, 27 are not fixed to one another in a manner which may not be dismantled but are simply forcibly mounted.

As a result, after use of the nasal secretion aspiration device 1, it is therefore possible to remove the used nasal end piece 2 from the hollow body 3, then to remove the first part 26 from the second part 27 in order to remove the impermeable membrane 8 from the part 26 and to discard it.

According to this embodiment of the invention, only the impermeable membrane 8 is for single use, the nasal end piece 2 having to be cleaned and sterilized after each use of the nasal secretion aspiration device.

It goes without saying that the invention is not limited to the embodiments disclosed above by way of example, but it nevertheless embraces all variants which fall within the scope of protection defined by the accompanying claims.

1. A nasal secretion aspiration device, wherein it comprises:
a hollow body defining a vacuum chamber able to be connected to a vacuum source, the vacuum chamber comprising an opening,
and a nasal end piece comprising a first open end adapted to the nostril, intended to be introduced into the nose of an individual and a second open end equipped with means for fixing in a leak-tight, removable manner in the opening of the hollow body, this second end being sealed by a non-porous element which may be deformed or displaced under the action of a vacuum created in the vacuum chamber by the vacuum source.

2. The nasal secretion aspiration device as claimed in claim 1, wherein the element sealing the second end of the nasal end piece comprises an elastically deformable impermeable membrane.

3. The nasal secretion aspiration device as claimed in claim 1, wherein the element sealing the second end of the nasal end piece comprises impermeable material.

4. The nasal secretion aspiration device as claimed in claim 1, wherein the second end of the nasal end piece is equipped with a cylindrical and axial tubular extension, inside of which is housed a piston which is displaceable under the action of a vacuum between a position located in the region of the second end and a position remote therefrom.

5. The nasal secretion aspiration device as claimed in claim 2, wherein the nasal end piece comprises two parts intended to be fixed to one another by nesting and wherein the impermeable membrane is fixed to the nasal end piece by clamping between the two parts fixed by nesting.

6. The nasal secretion aspiration device as claimed in claim 5, wherein the impermeable membrane comprises a peripheral flange intended to be retained in a housing formed between the two parts fixed by nesting.

7. The nasal secretion aspiration device as claimed in claim 6, wherein the peripheral flange is intended to cooperate with a retaining element formed on the external wall of one of the two parts forming the nasal end piece.

8. The nasal secretion aspiration device as claimed in claim 5, wherein the two parts forming the nasal end piece comprise means for nesting comprising a catch and a housing of corresponding shape in order to form a connection which may not be dismantled.

9. The nasal secretion aspiration device as claimed in claim 1, wherein the nasal end piece comprises at its first end a non-return valve avoiding the emptying of the nasal end piece when a vacuum ceases to be exerted on the deformable or displaceable element sealing the second end.

10. The nasal secretion aspiration device as claimed in claim 9, wherein the non-return valve comprises a ball maintained in abutment under the action of a spring against the opening of the first end, when no vacuum is created in the vacuum chamber.

11. The nasal section aspiration device as claimed in claim 1, wherein the means for fixing the nasal end piece to the hollow body are implemented by screwing.

12. The nasal secretion aspiration device as claimed in claim 1, wherein the means for fixing the nasal end piece to the hollow body are catch, nesting or bayonet type means.

13. The nasal secretion aspiration device as claimed in claim 1, wherein the hollow body contains an electric pump, driven by an electric motor, intended to create a vacuum in the vacuum chamber.

14. The nasal secretion aspiration device as claimed in claim 13, wherein the electric motor is supplied by batteries or an accumulator battery housed in the hollow body.

15. The nasal secretion aspiration device as claimed in claim 1, wherein the vacuum chamber is equipped with an aspiration end piece for mounting an aspiration tube, for example an oral aspiration tube.

16. The nasal secretion aspiration device as claimed in claim 1, wherein the vacuum chamber is defined by a wall capable of limiting the deformation of the deformable element sealing the second end of the nasal end piece.
